(19) United States
(12) Patent Application Publication
(10) Pub. No.: US 2015/0093369 A1
(43) Pub. Date: Apr. 2, 2015

(54) MIXTURE OF ENZYMES FROM ANTARCTIC KRILL FOR USE IN THE REMOVAL OF A BIOFILM

(71) Applicant: ARCIMBOLDO AB, Stockholm (SE)

(72) Inventors: Max Rutman, Santiago (CL); Wolfgang Vanscheidt, Freiburg (DE); Jan Vincent, Stockholm (SE); Frank Kochinke, San Jose, CA (US)

(73) Assignee: ARCIMBOLDO AB, Stockholm (SE)

(21) Appl. No.: 14/390,598

(22) PCT Filed: Mar. 15, 2013
(86) PCT No.: PCT/EP2013/055445

§ 371 Date: Oct. 3, 2014

Related U.S. Application Data
(60) Provisional application No. 61/620,893, filed on Apr. 5, 2012.

(51) Int. Cl. A61K 38/46; A61K 35/56

(52) U.S. Cl. CPC A61K 38/46; A61K 35/612
USPC 424/94.2

(57) ABSTRACT

The invention concerns a mixture of enzymes from krill for use in the treatment of a soft tissue including epithelial tissue and mucosa on which soft tissue a biofilm has formed or is expected to form, which soft tissue is a soft tissue of a mammal and which treatment comprises the removal of the biofilm or preventing a formation of the biofilm.

The invention concerns a mixture of enzymes from krill for use in the removal of a biofilm.

From Hahn Berg, I.C. et al., Eur. J. Oral. Sci. 2001, 109, pages 316-324, the proteolytic degradation of oral biofilms in the form of dental plaque, in vitro and in vivo, using proteases from Antarctic krill, referred to as “Krillase”, is known. Plaque is a very specific biofilm forming on the solid surface of teeth which biofilm comprises salivary proteins, a complex microbial community, and a matrix of polymers of bacterial and salivary origin.

From Hellgren, K., J. Clin. Dent. 2009, 20, pages 99-102, the use of a Krillase chewing gum for the reduction of gingivitis and dental plaque is known. According to this paper disruption of dental biofilm, together with limiting the bacterial attachment or adherence to oral surfaces, may represent a way to prevent plaque formation and consequently, most periodontal diseases.

US 2005/0025722 A1 discloses the use of a multifunctional enzyme that may be a krill-derived multifunctional enzyme. The enzyme may be used for the treatment of microbial infection such as an infection causing gastrointestinal disease, for the treatment of wound infection or for the treatment of acute or chronic inflammation.

Furthermore, it is known to use a krill enzyme mixture for debridement of wounds to accelerate healing of the wounds.

According to WO 2008/063229 A2 which concerns compositions to destroy internal cancerous lesions selectively by the administration of a combination of a debridement protease enzyme and a denaturant of cell structural proteins and/or cell adhesion proteins, enzymatic debridement involves the use of enzymes from outside the body to remove non-living tissue. Debridement enzymes cleave large immobile structural molecules of the cell into smaller molecules that can dissolve and migrate away.

The object of the present invention is to provide a further use for a mixture of enzymes from krill and a further method of treatment.

The object is solved by the features of claims 1 and 15. Useful embodiments are subject-matter of claims 2 to 14 and 16 to 26.

According to the invention a mixture of enzymes from krill for use in the treatment of a soft tissue including epithelial tissue and mucosa is provided. The soft tissue is a soft tissue of a mammal on which a biofilm has formed or is expected to form. If a biofilm has formed the treatment comprises the removal of the biofilm and if a biofilm is expected to form the treatment comprises preventing formation of the biofilm. The mixture of enzymes from krill is used in the removal or preventing the formation of the biofilm. Removal of the biofilm usually comprises contacting the biofilm with the mixture of enzymes. Preventing the formation of the biofilm usually comprises contacting a surface with the mixture of enzymes, on which surface the formation of the biofilm is expected to occur. The biofilm forming on soft tissue is different from the salivary proteins comprising biofilm forming plaque on teeth. This is in particular due to the specific surroundings and pH in the oral cavity. A biofilm is a film on a surface comprising specific proteins, other substances, microorganisms acting as a community and extracellular polymeric substances excreted by the microorganisms. A biofilm on a soft tissue usually comprises in addition to these constituents proteins and/or glycoproteins excreted from this soft tissue.

According to Rhoads, D. D. et al. “Evidence of Biofilms in Wounds and the Potential Ramifications”, BiofilmClub 2007, pages 1 to 15 biofilms with great species diversity form in wounds of compromised hosts that exhibit an impaired inflammatory response. The poor inflammatory response does not manage the wound's initial bacteria effectively. Consequently, the bacteria can persist within the wound and establish a biofilm community that not only can overcome the host's natural defenses but which is also recalcitrant to antibiotic therapy and other management strategies. The biofilm chronically maintains the wound in an inflammatory phase unless successful therapeutic means can help to eliminate the biofilm infection. The biofilm may even cause or support sepsis, in particular in burn wounds. The biofilm may form before or after a wound has been cleansed of bacteria and debris.

According to James G. A. et al. 2008, Wound Repair and Regeneration 16 (1), pages 37-44 a study comprising 50 chronic wound specimens and 16 acute wound specimens revealed that 60% of the chronic wound specimens and 6% of the acute wound specimens contained a biofilm. Molecular analyses of chronic wound specimens revealed diverse polymicrobial communities and the presence of bacteria, including strictly anaerobic bacteria, not revealed by culture.

The inventors surprisingly found that the biofilms forming on soft tissues can be removed by use of the mixture of enzymes from krill without destroying the soft tissues. They also found that the removal of the biofilm has several advantages in diseases and other conditions they found to be related to biofilms.

The mixture may comprise carbohydrases, lipolytic enzymes and/or proteases, in particular serine proteases, in particular serine proteases with trypsin-like activity or chymotrypsin-like activity, endopeptidases and/or exopeptidases, in particular carboxypeptidases or aminopeptidases. The mixture may be a crude extract from krill comprising proteolytic, lipolytic and carbohydrate splitting activity or an extract from krill more purified than the crude extract such that it has mainly proteolytic activity.

According to an embodiment of the invention the mixture of enzymes has at least two, in particular three, of a chymotrypsin-like, trypsin-like, endo-peptidase or exo-peptidase activity and/or the mixture of enzymes comprises enzymes having molecular weights in the range of 10 kD to 100 kD, in particular in the range of 15 kD to 60 kD, in particular in the range of 20 kD to 40 kD, as determined by SDS-PAGE.

The mixture may essentially consist of proteases comprising endopeptidases and exopeptidases.

The biofilm may be a biofilm causing an inflammatory or microbial condition of said mammal and the treatment may comprise a treatment of this condition.

According to one embodiment of the invention the soft tissue is a mucosa, in particular a mucosa damaged by aphtha or Herpes simplex virus infection or mucosa of the urinary tract or mucosa of the gastrointestinal tract, in particular oral mucosa, mucosa of the esophagus or mucosa of the stomach, in particular mucosa of the stomach affected by Helicobacter pylori. The soft tissue may also be an epithelium of skin, in particular of skin affected by a mycosis or a follicular unit. A follicular unit is a naturally-occurring hair follicle or grouping of two to four hair follicles that is extracted together with the hair(s) from a first area of a hair restoration patient to be implanted in a second area of the patient. The follicular unit may be treated with the mixture of enzymes before or even after transplantation. The soft tissue may also be a connective tissue on the surface of a graft, on the surface of a graft contact area of a transplant receipt and/or on the surface of a graft contact area of the graft. The graft may be a skin substitute, in particular a living skin substitute. The inventors found that a biofilm forms on said contact area and that removal of this biofilm prior to transplantation improves access of diverse growth factors and therewith improves the growing together of the graft and the transplant receipt. The graft may also be an organ to be transplanted. In the time between removal of the organ from the donor and inserting it in the recipient a biofilm may form. If a biofilms on such an organ is not removed prior to transplantation the biofilm may expand inside the body after transplantation, in particular due to the immunosuppression of a transplant receiving patient. The soft tissue may also be a connective tissue exposed by surgery. It may also be a chronic or an acute wound or a burn wound.

In another embodiment the soft tissue is the mucosa of the esophagus and the condition is reflux.

According to a further embodiment the soft tissue is oral mucosa and the condition is oral mucositis induced by radiation or chemotherapy.

The treatment may be by oral administration or by topical application, in particular by oral administration or by topical application of a sustained release formulation of the mixture.

The treatment may be by oral administration or by topical application of the mixture at least 4, in particular 6, in particular 7, in particular 8, times per day.

According to one further embodiment the treatment is a treatment for at least 3 days, in particular 4 days, in particular 5 days, in particular one week, in particular two weeks, or a treatment up to one month.

The casein-proteolytic activity of the mixture may be in the range of 0.0003 to 30, in particular in the range of 0.003 to 20, in particular in the range of 0.03 to 10, in particular in the range of 0.3 to 5, units per ml or per g of the mixture.

The krill may be Antarctic krill (Euphausia superba).

The invention further concerns a method of treatment of a soft tissue including epithelial tissue and mucosa which soft tissue is a soft tissue of a mammal, which treatment comprises the following steps:
Determining whether the soft tissue comprises a biofilm,
if it comprises a biofilm applying a mixture of enzymes from krill to an area of the soft tissue comprising said biofilm to contact the biofilm with the mixture and
maintaining the contact of the mixture with the biofilm for a time sufficient for a degradation of the biofilm.

The mixture may be a mixture as specified above. In one embodiment the biofilm is a biofilm causing an inflammatory or microbial condition of said mammal and the treatment comprises a treatment of this condition. The soft tissue may be a mucosa, in particular a mucosa damaged by aphtha or Herpes simplex virus infection or mucosa of the urinary tract or mucosa of the gastrointestinal tract, in particular oral mucosa, mucosa of the esophagus or mucosa of the stomach, in particular mucosa of the stomach affected by Helicobacter pylori, or an epithelium of skin, in particular of skin affected by a mycosis or a follicular unit, or a connective tissue on the surface of a graft, on the surface of a graft contact area of a transplant receipt and/or on the surface of a graft contact area of the graft or a connective tissue exposed by surgery or a chronic or an acute wound or a burn wound. The graft can be a skin substitute, in particular a living skin substitute.

In a particular embodiment of the method of treatment the soft tissue is the mucosa of the esophagus and the condition is reflux. In another particular embodiment the soft tissue is oral mucosa and the condition is oral mucositis induced by radiation or chemotherapy.

The mixture may be applied by oral administration or by topical application, in particular by oral administration or topical application of a sustained release formulation of the mixture. The mixture can be applied by oral administration or by topical application at least 4, in particular 6, in particular 7, in particular 8, times per day.

The treatment may be a treatment for at least 3 days, in particular 4 days, in particular 5 days, in particular one week, in particular two weeks, or a treatment up to one month.

In one embodiment of the method of treatment the casein-proteolytic activity of the mixture is in the range of 0.0003 to 30, in particular in the range of 0.003 to 20, in particular in the range of 0.03 to 10, in particular in the range of 0.3 to 5, units per ml or per g of the mixture. The krill may be Antarctic krill (Euphausia superba).

### EXAMPLES

### Example 1: Identification of Biofilms in Chronic Wounds by Scanning Electron Microscopy

A patient with a non-healing wound large enough to take several punch biopsies—at least 4 cm×5 cm—agrees to participate in the experiment. The study is geared towards determining the biofilm structure before and after application of the mixture of enzymes from krill via scanning electron microscopy.

The wound is photographed from a standardized view so that comparison photos over time can be taken. Then 2 wound samples at opposing sides of the wound are taken via punch biopsy and the samples are fixed in commonly accepted procedure. The fixation may be performed by incubation in 2.5% glutaraldehyde in 0.1 M phosphate-buffered solution (PBS), pH 7.2, followed by washing 3 times in PBS, and dehydration. The samples are stuck with double-sided tape to specimen stubs, followed by gold-platinum (50:50) ion coating. To improve the sample mounting the dorsal sides of the samples can be removed. Samples are visualized using a standard scanning electron microscope (SEM) that can be operated at a scanning voltage of 6 kV-10 kV. See also: James G. A. et al. 2008, Wound Repair and Regeneration 16 (1), pages 37-44.

Immediately following this initial “baseline” sampling the patient is scheduled to receive 5 applications of a solution of the mixture of enzymes from krill according to the invention in 2 hour intervals for 3 days, a total of 15 applications. The solution of the mixture of enzymes from krill to be used may be prepared as disclosed in EP 0 107 634 A2 or the standard Krillase® stock product purchased from Arcimboldo AB, Kraftriket 11, SE-104 05 Stockholm, Sweden as a vial containing 60 casein units of freeze-dried Euphausia extract powder and reconstituted in 10 ml sterile 0.9% sodium chloride solution. For the application sterile gauze is shaped according to the wound, soaked with the solution, and applied to the wound. The wound area is thereafter covered with occlusive film to prevent drying. After every 3rd application pictures are taken.

Two hours after the last application of the enzyme mixture solution 2 more punch biopsies are taken in proximity of first 2 biopsy areas. The samples are treated as described above.

Results: The first set of SEM pictures will clearly show a polymicrobial biofilm. The second set of pictures will show that the appearance of the wound is improving with increased enzyme mixture application number. The last set of SEM photographs will clearly show a clean wound and lack of organized biofilm. The patient will be admitted for a grafting procedure.

### Example 2: Histological Analysis

Two patients agree to participate in a study with the objective to determine biofilm existence via standard histological analysis before and after application of the mixture of enzymes according to the invention. Each of them has a non-healing wound large enough to take several punch biopsies (at least 4 cm×5 cm). The wounds of the 2 patients are photographed from a standardized view so that comparison photos over time can be taken. Then each wound of the two patients is sampled at opposing sides of the wound via punch biopsy. The excised samples are prepared for histological analysis by bisecting at their largest diameter for hematoxylin and eosin staining (HE). The tissues are then fixed in formalin, embedded in paraffin, and cut into 4-μm sections. Paraffin is removed with a xylene wash, followed by a standard hematoxylin and eosin staining protocol to prepare samples for analysis under a light microscope. The slides are examined using a digital analysis system by a masked observer evaluating all histological sections.

Immediately following this initial “baseline” sampling the patients are scheduled to receive 5 applications of a solution of the mixture of enzymes from krill according to the invention in 2 hour intervals for 3 days, a total of 15 applications. The solution of the mixture of enzymes from krill to be used may be the same as described for example 1. Application of the solution is as described for example 1. After every 3rd application pictures are taken.

Two hours after the last application of the enzyme mixture solution 2 more punch biopsies are taken in proximity of first 2 biopsy areas. The samples are treated as described above.

Results: The first set of slides for the first patient will clearly show a polymicrobial biofilm, while there will be no discernible bacterial colony for the second patient. The second set of pictures will show for both patients the appearance of the wound is improving with increasing enzyme mixture application number. In the case of the first patient a degradation of the biofilm and in addition the known debridement of the wound occurs whereas in the case of the second patient only the known debridement occurs. The last set of slides will show lack of organized biofilm for both patients and clean wounds.

### Example 3: Microbial Sequencing

Biofilms are polymicrobial and for antibiotic treatment therapy it is of value to know what the pathogens are and in what relative percentage they are in the infected area.

About 10 patients agree to participate to study wound bacteria compositions via DecodEX™ Microbial Sequencing offered by Pathogenius® Laboratories, 1004 Garfield Dr., Building #340, Lubbock, Tex. 79416, before and after application of the mixture of enzymes from krill according to the invention. Each of them has a non-healing wound large enough to take several punch biopsies (at least 4 cm×5 cm). The wounds of the 10 patients are photographed from a standardized view so that comparison photos over time can be taken. Then each wound of the patients is sampled following the instruction provided by Pathogenius Laboratories.

In general: 1. Obtain a sample using a curette or other debridement tools. 2. Place the sample (tissue or entire applicator tip) into the provided containers, 3. Send samples in provided shipment kit containers to Pathogenius Laboratories.

Immediately following this initial “baseline” sampling the patients are scheduled to receive 5 applications of a solution of the mixture of enzymes from krill according to the invention in 2 hour intervals for 3 days, a total of 15 applications. The solution of the mixture of enzymes from krill to be used may be the same as described for example 1. Application of the solution is as described for example 1. After every 3rd application pictures are taken.

Two hours after the last application of the enzyme mixture solution each patient's wound is sampled again in proximity of the first 2 sampling sites. The samples are treated as described above.

Results: A first set of tables will show that the majority of patients has a polymicrobial biofilm infection, however some patients do not show polymicrobial compositions. A following set of pictures will show for all patients that the appearance of the wound is improving with increasing enzyme mixture application number. A last set of tables will show that the degree of polymicrobial contamination of the wounds is drastically reduced or the polymicrobial contamination is eliminated.

### Example 4: Biofilms Identification via Swabbing and Microbial Sequencing

A patient with a small wound (anal fissure) that cannot be sampled by taking several biopsies out of the wound agrees to participate in the experiment. The study is geared towards determining the biofilm composition in a small wet wound before and after application of the mixture of enzymes from krill according to the invention via swabbing and microbial sequencing of the collected wound surface fluid.

“24-Hour Rapid Screening” sample kits from Pathogenius Laboratories will be used to examine the specimen for the most commonly identified microbes, including Staphylococcus (including MRSA), Streptococcus, Haemophilus, Enterococcus (including VRE), Pseudomonas, Serratia, Candida, and many more.

The patient is instructed to defecate and clean the area as unusual. Then 2 hours after the cleaning, the anal fissure of the patient will be photographed from a standardized view so that comparison photos over time can be taken. Then the anal mucosa wound surface of the patient is sampled following the instructions provided by Pathogenius Laboratories (common swabbing techniques). In general: 1. Obtain a sample using a sterile cotton-tipped applicator. 2. Place the sample entire applicator tip into the provided containers, 3. Send samples in provided shipment kit containers to Pathogenius Laboratories.

Immediately following this initial “baseline” sampling the patient is scheduled to receive 6 applications of a solution of the mixture of enzymes from krill according to the invention in 1 hour intervals. During that time the patient is not allowed to defecate. The solution of the mixture of enzymes from krill to be used may be the same as described for example 1. For the application sterile gauze is soaked with the solution and applied to the wound. After every 3rd application pictures are taken.

One hour after the last application of the enzyme mixture solution anal fissure wound is sampled again as described above.

Results: The first table will show the baseline bacterial composition of the wound, which is indicative of a polymicrobial biofilm infection. A following set of pictures will show that the appearance of the wound is improving with increasing enzyme mixture application number. A last table will show that the degree of the polymicrobial infection is drastically reduced as well as the number of bacterial species in the wound is lower than before.

### Example 5: Use of the Mixture of Enzymes from Krill According to the Invention for Preventing the Formation of a Biofilm on an Organ to be Transplanted

Immediately after removal of an organ from a donor which organ is intended to be transplanted a biofilm may form on its surface. To prevent formation of the biofilm the organ is put in a sterile 0.9% sodium chloride solution comprising the mixture of enzymes from krill according to the invention immediately after removal from the donor. To keep the organ vital it is kept in this solution at about 4° C. during its transport to the recipient. The enzymes from krill are predestinated for such an application because they are still active at 4° C. A further advantage is that it is not required to remove residual enzyme solution from the surface of the organ before transplantation because the enzymes are very quickly inactivated in the human body.

### Example 6: Use of the Mixture of Enzymes from Krill According to the Invention for Removal of a Helicobacter pylori (H. pylori) Biofilm

Resistance of H. pylori infection to conventional therapies is frequently associated with biofilm formation. Biofilms formed by H. pylory are complex microbiological ecosystems surrounding the bacteria and allowing proliferation in the hostile environment of the stomach. H. pylori biofilms on human gastric mucosa has been proven to be the causative agent of gastric ulcers.

To degrade the H. pylori biofilm patients are treated for 2 days with omeprazole to normalize their gastric pH. Thereafter treatment with omeprazole is maintained and in addition patients are treated with the mixture of enzymes from krill according to the invention by administration of gelatin capsule comprising the mixture of enzymes 3 times a day for 7 days. Biofilm removal is followed in parallel by sampling of gastric biopsies and examination of the biopsies for the presence of the biofilm. Examination may be performed by scanning electron microscopy as described in example 1.

### Example 7: Use of the Mixture of Enzymes from Krill According to the Invention for the Treatment of Follicular Units After Transplantation

A patient's skin area with freshly transplanted follicular units is treated by applying the mixture of enzymes from krill according to the invention. The area comprises a small wound at every site where a follicular unit has been inserted. A solution of the mixture of enzymes from krill to be used may be the same as described for example 1. For the application sterile gauze is soaked with the solution and applied to the area 3 times per day for 7 days. Due to this application biofilms formed at the follicular units and/or the wounds are removed and/or the formation of biofilm at the follicular units and/or the wounds is prevented resulting in a relatively fast healing without significant scar formation and without significant loss of freshly implanted hair.

1. Mixture of enzymes from krill for use in the treatment of a soft tissue including epithelial tissue and mucosa on which soft tissue a biofilm has formed or is expected to form, which soft tissue is a soft tissue of a mammal and which treatment comprises the removal of the biofilm or preventing a formation of the biofilm.

2. Mixture according to claim 1, wherein the mixture comprises carbohydrases, lipolytic enzymes and/or proteases, in particular serine proteases, in particular serine proteases with trypsin-like activity or chymotrypsin-like activity, endopeptidases and/or exopeptidases, in particular carboxypeptidases or aminopeptidases.

3. Mixture according to any of the preceding claims, wherein the mixture of enzymes has at least two, in particular three, of a chymotrypsin-like, trypsin-like, endo-peptidase or exo-peptidase activity and/or wherein the mixture of enzymes comprises enzymes having molecular weights in the range of 10 kD to 100 kD, in particular in the range of 15 kD to 60 kD, in particular in the range of 20 kD to 40 kD, as determined by SDS-PAGE.

4. Mixture according to any of the preceding claims, wherein the mixture essentially consists of proteases comprising endopeptidases and exopeptidases.

5. Mixture according to any of the preceding claims, wherein the biofilm is a biofilm causing an inflammatory or microbial condition of said mammal and the treatment comprises a treatment of this condition.

6. Mixture according to any of the preceding claims, wherein the soft tissue is a mucosa, in particular a mucosa damaged by aphtha or Herpes simplex virus infection or mucosa of the urinary tract or mucosa of the gastrointestinal tract, in particular oral mucosa, mucosa of the esophagus or mucosa of the stomach, in particular mucosa of the stomach affected by Helicobacter pylori, or an epithelium of skin, in particular of skin affected by a mycosis or a follicular unit, or a connective tissue on the surface of a graft, on the surface of a graft contact area of a transplant receipt and/or on the surface of a graft contact area of the graft or a connective tissue exposed by surgery or a chronic or an acute wound or a burn wound.

7. Mixture according to claim 6, wherein the graft is a skin substitute, in particular a living skin substitute.

8. Mixture according to claim 6, wherein the soft tissue is the mucosa of the esophagus and wherein the condition is reflux.

9. Mixture according to claim 6, wherein the soft tissue is oral mucosa and the condition is oral mucositis induced by radiation or chemotherapy.

10. Mixture according to any of the preceding claims, wherein the treatment is by oral administration or by topical application, in particular by oral administration or topical application of a sustained release formulation of the mixture.

11. Mixture according to claim 10, wherein the treatment is by oral administration or by topical application of the mixture at least 4, in particular 6, in particular 7, in particular 8, times per day.

12. Mixture according to any of the preceding claims, wherein the treatment is a treatment for at least 3 days, in particular 4 days, in particular 5 days, in particular one week, in particular two weeks, or a treatment up to one month.

13. Mixture according to any of the preceding claims, wherein the casein-proteolytic activity of the mixture is in the range of 0.0003 to 30, in particular in the range of 0.003 to 20, in particular in the range of 0.03 to 10, in particular in the range of 0.3 to 5, units per ml or per g of the mixture.

14. Mixture according to any of the preceding claims, wherein the krill is Antarctic krill (Euphausia superba).

15. Method of treatment of a soft tissue including epithelial tissue and mucosa which soft tissue is a soft tissue of a mammal, which treatment comprises the following steps:
Determining whether the soft tissue comprises a biofilm,
if it comprises a biofilm applying a mixture of enzymes from krill to an area of the soft tissue comprising said biofilm to contact the biofilm with the mixture and
maintaining the contact of the mixture with the biofilm for a time sufficient for a degradation of the biofilm.

16. Method of treatment according to claim 15, wherein the mixture is a mixture as specified in any of claims 2 to 4.

17. Method of treatment according to claim 15 or 16, wherein the biofilm is a biofilm causing an inflammatory or microbial condition of said mammal and the treatment comprises a treatment of this condition.

18. Method of treatment according to any of claims 15 to 17, wherein the soft tissue is a mucosa, in particular a mucosa damaged by aphtha or Herpes simplex virus infection or mucosa of the urinary tract or mucosa of the gastrointestinal tract, in particular oral mucosa, mucosa of the esophagus or mucosa of the stomach, in particular mucosa of the stomach affected by Helicobacter pylori, or an epithelium of skin, in particular of skin affected by a mycosis or a follicular unit, or a connective tissue on the surface of a graft, on the surface of a graft contact area of a transplant receipt and/or on the surface of a graft contact area of the graft or a connective tissue exposed by surgery or a chronic or an acute wound or a burn wound.

19. Method of treatment according to claim 18, wherein the graft is a skin substitute, in particular a living skin substitute.

20. Method of treatment according to claim 18, wherein the soft tissue is the mucosa of the esophagus and wherein the condition is reflux.

21. Method of treatment according to claim 18, wherein the soft tissue is oral mucosa and the condition is oral mucositis induced by radiation or chemotherapy.

22. Method of treatment according to any of claims 15 to 21, wherein the mixture is applied by oral administration or by topical application, in particular by oral administration or topical application of a sustained release formulation of the mixture.

23. Method of treatment according to claim 22, wherein the mixture is applied by oral administration or by topical application at least 4, in particular 6, in particular 7, in particular 8, times per day.

24. Method of treatment according to any of claims 15 to 23, wherein the treatment is a treatment for at least 3 days, in particular 4 days, in particular 5 days, in particular one week, in particular two weeks, or a treatment up to one month.

25. Method of treatment according to any of claims 15 to 24, wherein the casein-proteolytic activity of the mixture is in the range of 0.0003 to 30, in particular in the range of 0.003 to 20, in particular in the range of 0.03 to 10, in particular in the range of 0.3 to 5, units per ml or per g of the mixture.

26. Method of treatment according to any of claims 15 to 25, wherein the krill is Antarctic krill (Euphausia superba).
